(19) United States
(12) Patent Application Publication
(10) Pub. No.: US 2023/0389932 A1
(43) Pub. Date: Dec. 7, 2023

(54) CLOT TREATMENT SYSTEMS, SUCH AS FOR USE IN REMOVING CLOT MATERIAL FROM A LEFT ATRIAL APPENDAGE (LAA), AND ASSOCIATED DEVICES AND METHODS

(71) Applicant: Inari Medical, Inc., Irvine, CA (US)

(72) Inventors: Parker Ozenne, Aliso Viejo, CA (US); Jared Shimizu, Tustin, CA (US); Benjamin Edward Merritt, San Clemente, CA (US); Hamid Rafi, Irvine, CA (US)

(21) Appl. No.: 18/329,890

(22) Filed: Jun. 6, 2023

Related U.S. Application Data
(60) Provisional application No. 63/349,969, filed on Jun. 7, 2022.

(51) Int. Cl. A61B 17/12; A61B 17/00

(52) U.S. Cl. CPC A61B 17/12122; A61B 17/0057; A61B 17/12172; A61B 2017/00575

(57) ABSTRACT

Disclosed herein are clot treatment systems and associated devices and methods. In some embodiments, a clot treatment system includes a catheter having a shaped distal portion configured to access a patient's left atrial appendage. The shaped distal portion of the catheter can be aligned with and/or positioned within the patient's left atrial appendage and used to aspirate clot material therefrom. The clot treatment system can further include a clot capture device configured to at least partially cover an opening of the left atrial appendage. For example, the clot capture device can be aligned with, positioned against, and/or inserted at least partially through an ostium of the patient's left atrial appendage to catch any clot material that breaks free from the left atrial appendage.

### CROSS-REFERENCE TO RELATED APPLICATION

This application claims the benefit of U.S. Patent Application No. 63/349,969, filed Jun. 7, 2022, and titled “CLOT TREATMENT SYSTEMS, SUCH AS FOR USE IN REMOVING CLOT MATERIAL FROM A LEFT ATRIAL APPENDAGE (LAA), AND ASSOCIATED DEVICES AND METHODS,” which is incorporated herein by reference in its entirety.

### TECHNICAL FIELD

The present technology generally relates to clot treatment systems, including clot treatment systems for use in removing clot material from a left atrial appendage, and associated devices and methods.

### BACKGROUND

Thrombosis is the local coagulation or clotting of the blood in a part of the circulatory system, and a thrombus is a blood clot formed in situ within the vascular system. An intracardiac thrombus is a blood clot that forms in a patient's heart. One example of an intracardiac thrombus is a left atrial appendage LAA thrombus, which is a blood clot that forms within the left atrial appendage of the heart. If an intracardiac thrombus (e.g., an LAA thrombus) breaks off (embolizes) and flows towards the limbs or brain, it can lead to organ failure, acute limb ischemia (ALI) and/or a stroke.

Additionally, the presence of intracardiac thrombi can adversely affect (e.g., contraindicate) many structural heart procedures, such as ablations, left atrial appendage closure (LAAC), mitral valve repair/replacement (TMVR), aortic valve repair/replacement (TAVR), and patent foramen ovale (PFO) closure. When an intracardiac thrombus is detected, the heart procedure is typically canceled, and the patient is often instructed to take an oral anticoagulant for about 4-6 weeks to attempt to break down the intracardiac thrombus. However, there can be instances where the thrombus is still not fully resolved after this 4-6 week period. In this case, the physician might decide to proceed with the procedure with thrombus present or wait even longer, prescribing a different anticoagulant.

Taking an oral anticoagulant can have several risks, such as an increased likelihood of excessive bleeding or hemorrhage. Additionally, patients could be contraindicated for anticoagulants because of their side effects, making it difficult to remove these patients' intracardiac thrombi. Moreover, postponing a procedure has its own risks such as an increased risk of mortality for some procedures. Additionally, patients may not always take prescribed anticoagulants, which would lead to the clot not resolving and/or further delays to the heart procedure.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A is a partially schematic side view of a clot treatment system configured in accordance with embodiments of the present technology.

FIG. 1B is an end view of a shaped portion of the clot treatment system of FIG. 1A in accordance with embodiments of the present technology.

FIG. 2 is a side cross-sectional view of the shaped portion of the clot treatment system of FIGS. 1A and 1B inserted into a human heart in accordance with embodiments of the present technology.

FIGS. 3, 4, 5A, 5B, 6A, 6B, and 6C are side views of respective clot capture and/or embolic protection devices configured in accordance with embodiments of the present technology.

FIG. 7 is a side view of a clot treatment device positioned within a left atrial appendage in accordance with embodiments of the present technology.

FIG. 8 is a side cross-sectional view of a heart of a patient and a clot capture and/or embolic protection device positioned downstream from the heart in accordance with embodiments of the present technology.

FIG. 9 is a side cross-sectional view of a heart of a patient with the shaped portion of FIG. 1B positioned within the left atrium, in accordance with embodiments of the present technology.

FIG. 10 is a side cross-sectional view of a heart of a patient with the clot capture device of FIG. 3 positioned in accordance with embodiments of the present technology.

### DETAILED DESCRIPTION

The present technology is generally directed to clot treatment systems, and associated devices and methods. In some embodiments, a representative clot treatment system includes a catheter having a shaped distal portion configured to facilitate insertion of the catheter to specific portions of a patient's anatomy. For example, the shaped portion can be curved to facilitate access to the interior of the patient's left atrial appendage. The clot treatment system can further include a pressure source fluidly coupled to the catheter and configured to apply negative pressure to the catheter to aspirate clot material from within the patient. Accordingly, the shaped portion of the catheter can be aligned with and/or positioned within the patient's left atrial appendage and used to aspirate clot material from within the left atrial appendage. In some aspects of the present technology, the clot treatment systems provides for the mechanical removal of clot material from the left atrial appendage—thereby reducing or eliminating the need to cancel a structural heart procedure if clot material is discovered within a patient's left atrial appendage, reducing or eliminating the need to prescribe anticoagulants to treat the clot material, and/or increasing the speed with which the structural heart procedure can be performed.

In some embodiments, the clot treatment system further includes a clot capture and/or embolic protection device (e.g., a funnel) configured to inhibit movement or migration of clot material away from the left atrial appendage, such as by at least partially or fully preventing the clot material from embolizing. For example, the clot capture device can be aligned with, positioned against, and/or inserted at least partially through the ostium of the patient's left atrial appendage such that the clot capture device “catches” part or all of any clot material that breaks free from the left atrial appendage. Accordingly, the clot capture device can reduce the risk that clot material within the left atrial appendage embolizes and moves through the heart and vasculature of the patient. In some embodiments, the clot capture device can include a funnel configured to (i) allow blood or other fluid within the left atrial appendage to flow through the funnel and (ii) at least partially prevent the clot material within the left atrial appendage from leaving the left atrial appendage. Additionally, or alternatively, the clot capture device can be positioned downstream from the left atrial appendage, such as within the patient's aorta, to capture any of the clot material flowing therethrough. In these and other embodiments, the clot treatment system can further include a coring element or other clot treatment device that can be inserted into the left atrial appendage to engage and/or remove at least a portion of the clot material positioned therein.

Although certain aspects of the present technology are described with reference to clot treatment and/or removal procedures associated with a left atrial appendage of a patient, a person of ordinary skill in the art will appreciate that the present technology can be used to treat and/or remove clots in other portions of the patient anatomy. For example, at least some embodiments of the present technology can be used to treat and/or remove clots in other locations within the left side of the heart, such as within the left atrium, the left ventricle, and/or the mitral valve.

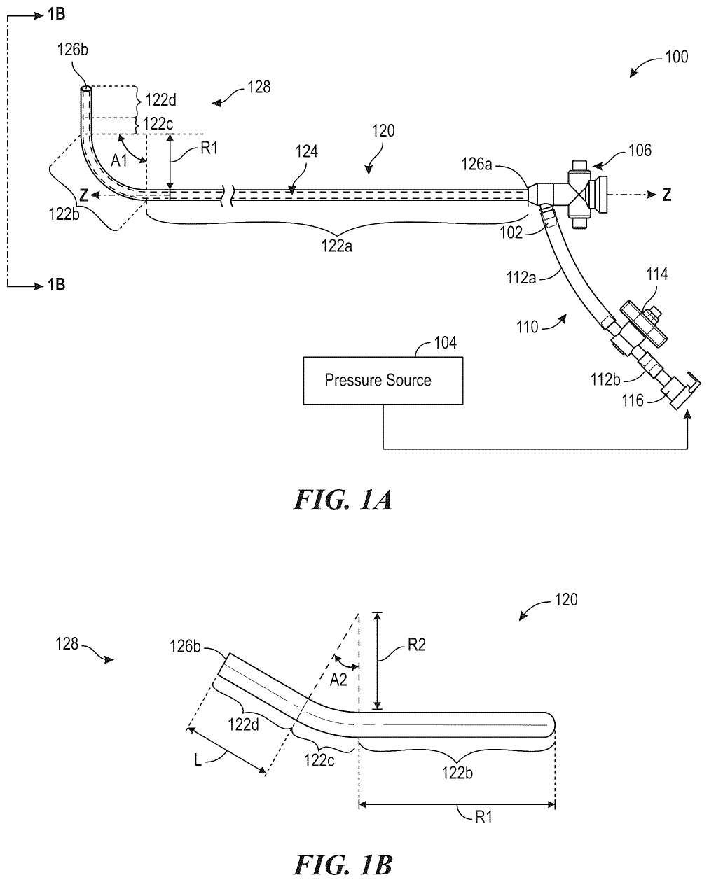

Certain details are set forth in the following description and in FIGS. 1A-10 to provide a thorough understanding of various embodiments of the present technology. In other instances, well-known structures, materials, operations, and/or systems often associated with intravascular procedures, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, element 110 is first introduced and discussed with reference to FIG. 1A.

To the extent any materials incorporated herein by reference conflict with the present disclosure, the present disclosure controls.

FIG. 1A is a partially schematic side view of a clot treatment system 100 (“the system 100”) configured in accordance with embodiments of the present technology. The system 100 can also be referred to as an aspiration assembly, a vascular access system, a clot removal system, a thrombectomy system, a left atrial appendage (“LAA”) thrombectomy system, and/or the like. In the illustrated embodiment, the system 100 includes a tubing assembly 110 fluidly coupled to a catheter 120 via a valve 106. The catheter 120 is in a relaxed, unconstrained configuration in FIG. 1A. In some embodiments, the catheter 120 is an elongate member configured to be inserted into and through a patient's vasculature and used to, for example, treat clot material therein. In other embodiments, the catheter 120 can be an introducer sheath configured to be inserted through the skin and tissue tract of the patient to provide an access site through which other components (e.g., other catheters used to treat clot material) can traverse to easily access the vasculature. Accordingly, while referred to as “catheter 120,” the catheter 120 can comprise an introducer sheath, an access sheath, and/or another type of elongate member configured to be inserted through the skin and tissue tract and/or to traverse the vasculature of a patient. In general, the system 100 (i) can include features generally similar in structure and/or function, or identical in structure and/or function, to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter 120 includes (i) a proximal region or portion 122a, (ii) an intermediate region 122b adjacent to and distal of the proximal region 122a, (iii) a distal region 122c adjacent to and distal of the intermediate region 122b, and (iv) a distal tip region 122d adjacent to and distal of the distal region 122c. In some embodiments, the catheter 120 has an outer diameter of at least 20 Fr, such as at least 21 Fr, 22 Fr, 23 Fr, 24 Fr, etc., up to, e.g., 30 Fr. In other embodiments, however, the catheter 120 has an outer diameter of less than 20 Fr, or another suitable outer diameter. The catheter 120 further defines a lumen 124 (shown using dashed-line in FIG. 1A) extending entirely therethrough from the proximal region 122a to the distal tip region 122d. In some embodiments, the lumen 124 has a diameter (e.g., corresponding to the inner diameter of the catheter 120) of at least 15 Fr, such as at least 16 Fr, 17 Fr, 18 Fr, 19 Fr, 20 Fr, 21 Fr, etc., up to, e.g., 25 Fr. For example, the lumen 124 can have a diameter of 20.6 Fr. In other embodiments, however, the lumen 124 can have a diameter less than 15 Fr, or another suitable diameter. The proximal region 122a defines a proximal terminus 126a of the catheter 120, and the distal tip region 122d defines a distal terminus 126b of the catheter 120.

The proximal region 122a can be least generally linear and define a longitudinal axis Z. The intermediate region 122b, the distal region 122c, and/or the distal tip region 122d can define a shaped distal portion 128 (“shaped portion 128”) that is curved relative to the longitudinal axis Z. FIG. 1B is an end view of the shaped portion 128 taken along line 1B-1B in FIG. 1A. Referring to FIGS. 1A and 1B together, the intermediate region 122b can be curved relative to the longitudinal axis Z about a first radius R1 at a first angle A1, and the distal region 122c can be curved relative to the longitudinal axis Z about a second radius R2 at a second angle A2 (as shown in FIG. 1B). The first radius R1 can be in a first plane that includes the longitudinal axis Z, and the second radius R2 can be in a second plane that is different than (e.g., perpendicular to, not parallel to, angled relative to, and the like) the first plane. In the illustrated embodiment, the first radius R1 is 2 inches, the first angle A1 is 90 degrees, the second radius R2 is 1.25 inches, and the second angle A2 is 30 degrees. In other embodiments, the first radius R1 can be between about 0.5-5.0 inches, the first angle A1 can be between about 60-120 degrees, the second radius R2 can be between about 0.5-2.5 inches, and the second angle A2 can be between about 10-60 degrees. The distal tip region 122d can be at least generally linear and can extend a length L from the distal region 122c. In the illustrated embodiment, the length L is 1.0 inch. In other embodiments, the length L can be between about 0.25 inch and about 2 inches, such as at least 0.25 inch, 0.5 inch, 1.25 inch, or another suitable length. One or more of the first radius R1, the second radius R2, the first angle A1, and/or the second angle A2 can vary based, at least in part, on whether a dilator is positioned within the catheter 120 (e.g., within the shaped portion 128 of the catheter 120). In at least some embodiments, one or more of the first radius R1, the second radius R2, the first angle A1, and/or the second angle A2 can have a respective first value without a dilator positioned within the shaped portion 128 and a second value different than (e.g., greater or less than) the first value with the dilator positioned within the shaped portion 128. For example, the first radius R1 can be 2 inches without the dilator and 2.1 inches with the dilator, the first angle A1 can be 90 degrees without the dilator and 85 degrees with the dilator, the second radius R2 can be 1 inch without the dilator and 2 inches with the dilator, and/or the second angle A2 can be 30 degrees without the dilator and 15 degrees with the dilator. In other embodiments, individual ones of the first radius R1, the second radius R2, the first angle A1, and/or the second angle A2 can change between any two of the other values described previously herein.

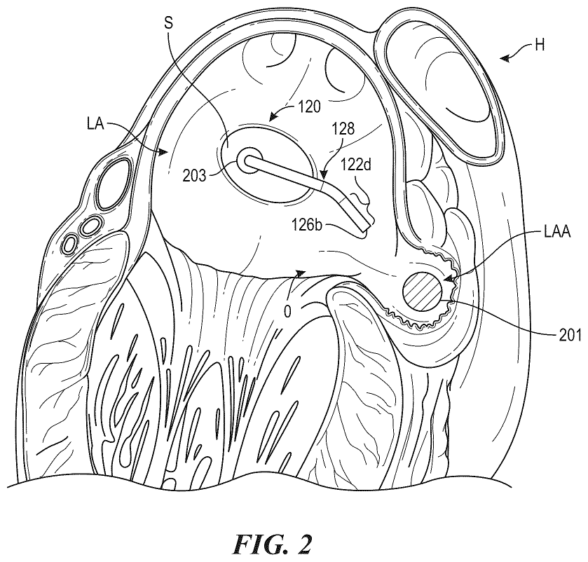

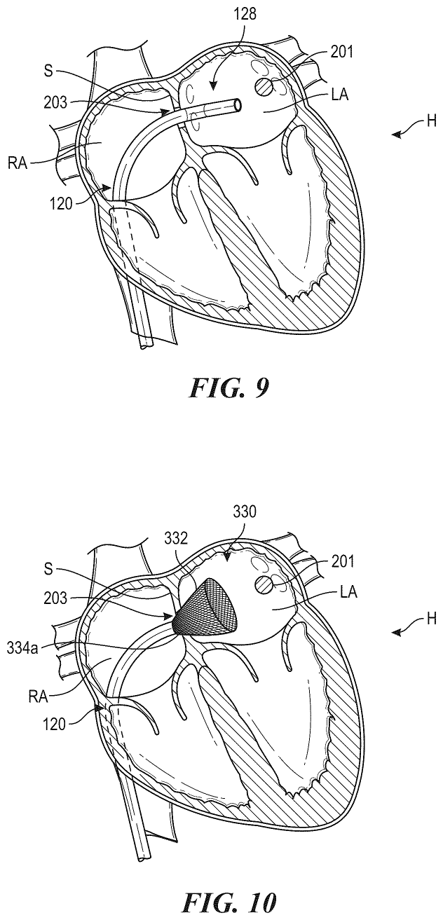

In some aspects of the present technology, the curvature of the shaped portion 128 is expected to improve access to select portions of a patient's anatomy, such as the left atrial appendage, as described in detail below with reference to FIG. 2, and/or the left atrium, as described in detail below with reference to FIGS. 9 and 10. Although the shaped portion 128 has specific dimensions and/or curvature in the embodiments illustrated in FIGS. 1A and 1B, in other embodiments the shaped portion 128 can have other dimensions and/or curvature to, for example, facilitate placement of the distal tip region 122d proximate a desired position in the heart of the patient. For example, although in the illustrated embodiment the catheter 120 includes two curved regions (e.g., the intermediate region 122b and the distal region 122c) between the proximal terminus 126a and the distal terminus 126b, in other embodiments the catheter 120 can include more or fewer curved regions. In at least some embodiments, for example, the shaped portion 128 can include at least one, three, four, or another suitable number of curved regions, individual ones of which can have a same or different curvature as the intermediate region 122b and/or the distal region 122c. In these and other embodiments, one or both of the proximal region 122a and/or the distal tip region 122d can be curved and/or otherwise configured to deflect away from a generally linear configuration, as described in further detail below.

The catheter 120 can have varying lengths, flexibilities, shapes, thicknesses, and/or other properties in/along the various regions 122a-d. For example, the catheter 120 can comprise one or more coils, braids, and/or other structures positioned between one or more liner layers (e.g., inner and outer liner layers). In some embodiments, the catheter 120 can include several features generally similar or identical in structure and/or function to any of the catheters described in U.S. patent application Ser. No. 17/529,018, titled “CATHETERS HAVING SHAPED DISTAL PORTIONS, AND ASSOCIATED SYSTEMS AND METHODS,” and filed Nov. 17, 2021, and/or U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, each of which is incorporated by reference herein in its entirety.

In some embodiments, the shaped portion 128 can be configured to move between (i) the relaxed configuration illustrated in FIGS. 1A and 1B in which the shaped portion 128 has the curved shape and (ii) a constrained position in which the shaped portion 128 is more closely aligned with the longitudinal axis Z. For example, in the relaxed configuration, the intermediate region 122b and/or the distal region 122c can be configured (e.g., heat set) to deflect away from the longitudinal axis Z of the catheter 120 relative to the proximal region 122a. In the constrained configuration, the intermediate region 122b and/or the distal region 122c can be configured to deflect toward the longitudinal axis Z so that one or more of the intermediate region 122b, the distal region 122c, and/or the distal tip region 122d are at least generally aligned (e.g., colinear) with the proximal region 122a and/or the longitudinal axis Z.

The valve 106 is fluidly coupled to the lumen 124 of the catheter 120 and can be integral with or coupled to the proximal region 122a of the catheter 120 such that these components move together. In some embodiments, the valve 106 is a hemostasis valve that is configured to maintain hemostasis during a clot treatment procedure by preventing fluid flow in a proximal direction P through the valve 106 as various components such as dilators, delivery sheaths, pull members, guidewires, interventional devices, other aspiration catheters, and so on are inserted through the valve 106 to be delivered through the catheter 120 to a treatment site in a blood vessel. The valve 106 can include a branch or side port 102 configured to fluidly couple the lumen 124 of the catheter 120 to the tubing assembly 110. In some embodiments, the valve 106 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 104, such as a syringe. The pressure source 104 can be configured to generate (e.g., form, create, charge, build-up) a vacuum (e.g., negative relative pressure) and store the vacuum for subsequent application to the catheter 120. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 104 and/or other suitable components. In some embodiments, the fluid control device 114 is a stopcock that is fluidly coupled to (i) the side port 102 of the valve 106 via the first tubing section 112a and (ii) the connector 116 via the second tubing section 112b. The fluid control device 114 is externally operable by a user to regulate the flow of fluid therethrough and, specifically, from the lumen 124 of the catheter 120 to the pressure source 104. In some embodiments, the connector 116 is a quick-release connector (e.g., a quick disconnect fitting) that enables rapid coupling/decoupling of the catheter 120 and the fluid control device 114 to/from the pressure source 104.

During a clot treatment procedure, at least a portion of the system 100, such as the distal terminus 126b and/or distal tip region 122d of the catheter 120, can be inserted through the vasculature of a patient. In some embodiments, the system 100 is inserted through an introducer sheath that traverses the skin and tissue of the patient to provide an access site. When the catheter 120 is positioned at a target treatment location proximate to clot material (e.g., a left atrial appendage thrombus, pulmonary embolism, deep vein thrombosis, and the like) within the patient, a user can first close the fluid control device 114 before generating a vacuum in the pressure source 104 by, for example, withdrawing the plunger of a syringe coupled to the connector 116. In this manner, a vacuum is charged within the pressure source 104 (e.g., a negative pressure is maintained) before the pressure source 104 is fluidly connected to the lumen 124 of the catheter 120. To aspirate the lumen 124 of the catheter 120, the user can open the fluid control device 114 to fluidly connect the pressure source 104 to the catheter 120 and thereby apply or release the vacuum stored in the pressure source 104 to the lumen 124 of the catheter 120. Opening of the fluid control device 114 instantaneously or nearly instantaneously applies the stored vacuum pressure to the tubing assembly 110 and the catheter 120, thereby generating a suction pulse throughout the catheter 120 that can aspirate the clot material into the catheter 120. In particular, the suction is applied at the distal tip region 122d of the catheter 120 to suck/aspirate at least a portion of the clot material proximate the distal tip region 122d into the lumen 124 of the catheter 120. Additionally, or alternatively, the catheter 120 can act as an introducer sheath and can be inserted through the skin and tissue of a patient and partially into a vessel to provide an access point through which other medical instruments can be delivered and/or otherwise used to treat the patient. In these and other embodiments, the user can generate the vacuum in the pressure source 104 while the fluid control device 114 is open (e.g., while the pressure source 104 is fluidly connected to the lumen 124 of the catheter 120) to thereby aspirate the clot material in concert with and/or while also simultaneously generating the vacuum, e.g., without or substantially without storing the vacuum in the pressure source 104.

FIG. 2 is a side cross-sectional view of the shaped distal portion 128 of the clot treatment system 100 of FIGS. 1A and 1B inserted into a human heart H (“the heart H”) in accordance with embodiments of the present technology. The heart H includes a left atrium LA, and the left atrium LA includes a left atrial appendage LAA. The left atrial appendage LAA can be fluidly coupled to the left atrium LA by an ostium O, such that fluid can flow between the left atrium LA and the left atrial appendage LAA. In some patients, blood and/or other clot material 201 (shown schematically) can accumulate within the left atrial appendage LAA and form a clot or thrombus therein. Referring to FIGS. 1A, 1B, and 2 together, in the illustrated embodiment the catheter 120 has been inserted into and advanced through a right atrial vein (not shown) of the patient and has entered the left atrium LA transeptally via an opening 203 formed in the septum S between the left atrium LA and the right atrium of the heart H (not shown).

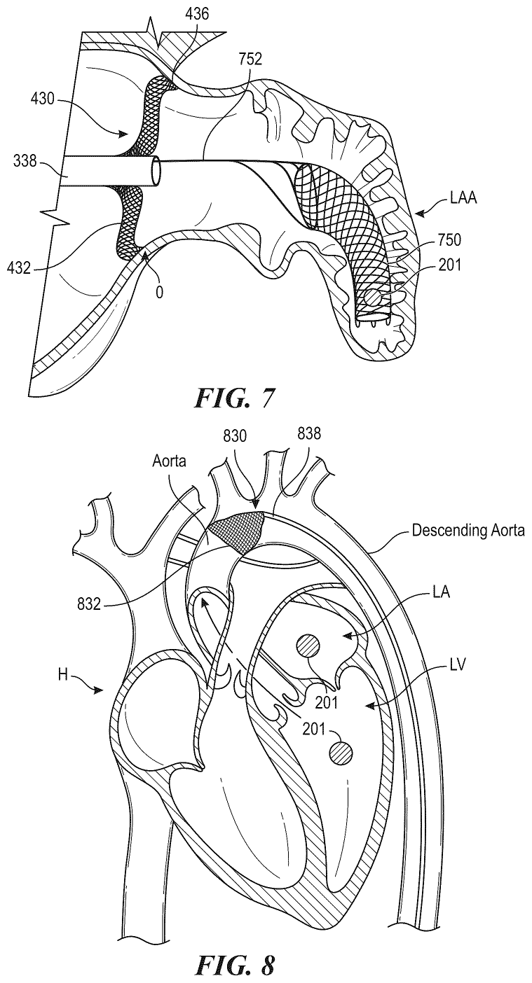

The shaped portion 128 of the catheter 120 is curved such that the distal tip region 122d can be aligned with the left atrial appendage LAA. For example, the distal terminus 126b can be positioned proximate the ostium O and/or extend at least partially though the ostium O into the left atrial appendage LAA. When so positioned, the catheter 120 can be used to aspirate the clot material 201 from the left atrial appendage LAA as described in detail above with reference to FIGS. 1A and 1B. In some embodiments, one or more clot treatment and/or removal devices, such as the clot treatment device of FIG. 7, can be inserted through the catheter 120 to assist with the treatment of the clot material 201.

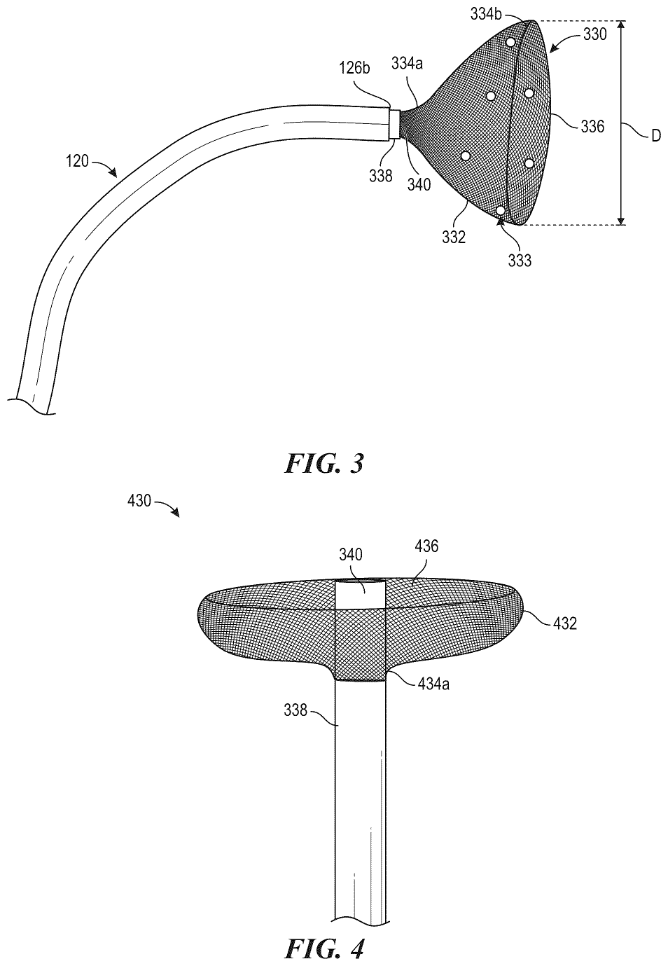

FIGS. 3-6C are side views of respective clot capture and/or embolic protection devices 330, 430, 530a, 530b, 630 (collectively referred to as “devices 330-630”) configured in accordance with embodiments of the present technology. In some embodiments, one or more of the devices 330-630 can be used in the clot treatment system 100 described in detail with reference to FIGS. 1A-2. For example, the devices 330-630 can be coupled to the catheter 120 or advanced through the catheter 120. At least some aspects of one or more of the devices 330-630 can be generally similar or identical in structure and/or function to one or more of the other devices 330-630. Accordingly, like names and/or reference numbers are used to indicate aspects of the devices 330-630 that can be generally similar or identical.

Referring to FIG. 3, the device 330 includes a funnel 332 having a proximal end portion 334a and a distal end portion 334b. The distal end portion 334b can define a distal edge or terminus 336 of the funnel 332. Referring to FIGS. 2 and 3 together, the distal edge 336 can be configured to contact or otherwise engage at least a portion of the patient's left atrial appendage LAA, such as the ostium O and/or one or more interior surfaces of the left atrial appendage LAA. The distal end portion 334b and/or the distal edge 336 can define an outer dimension D (e.g., a diameter, a width) of the funnel 332. The outer dimension D can be between about 15 mm to about 50 mm, such as at least 35 mm, or another value therebetween. In these and other embodiments, the outer dimension D can be equal to or greater than a corresponding inner dimension of the left atrial appendage LAA, such as a diameter of the ostium O. The proximal end portion 334a can be coupled to a distal tip 340 of an elongate member 338, such as a shaft or catheter. In the illustrated embodiment, the elongate member 338 is positioned within the catheter 120 with the funnel 332 extending distally beyond the distal terminus 126b of the catheter 120. In some embodiments, the elongate member 338 can be shaped and/or steerable. For example, the elongate member 338 can include at least some aspects that are generally similar or identical in structure and/or function to one or more of the catheters described in U.S. patent application Ser. No. 17/529,018, titled “CATHETERS HAVING SHAPED DISTAL PORTIONS, AND ASSOCIATED SYSTEMS AND METHODS,” and filed Nov. 17, 2021, and/or U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, each of which is incorporated by reference herein in its entirety.

The funnel 332 can be configured to transition between an expanded state (shown in FIG. 3) and a collapsed or low-profile delivery state. In the illustrated embodiment, the elongate member 338 can be moved relative to the catheter 120 to transition the funnel 332 between the expanded state and the collapsed state. For example, the elongate member 338 can be retracted proximally within the catheter 120 to withdraw the funnel 332 toward the distal terminus 126b of the catheter 120 and/or into the catheter 120 to transition the funnel 332 from the expanded state to the collapsed state. Additionally, or alternatively, the catheter 120 can be advanced proximally over the elongate member 338 to drive the distal terminus 126b toward and/or against the funnel 332 and transition the funnel 332 from the expanded state to the collapsed state. To transition the funnel 332 from the collapsed state to the expanded state, the catheter 120 can be retracted proximally relative to the elongate member 338 to uncover the funnel 332 and allow the funnel 332 to expand toward and/or to the expanded state. Additionally, or alternatively, the elongate member 338 can be advanced distally from within the catheter 120 to extend distally beyond the distal terminus 126b to thereby uncover the funnel 332 from within the catheter 120 and allow the funnel 332 to expand from the collapsed state to the expanded state.

The funnel 332 can include a plurality of braided filaments, such as a plurality of shape memory wires heat set to expand to the expanded state shown in FIG. 3. The funnel 332 can further define one or more openings or pores 333 (shown schematically in FIG. 3). Although eight pores 333 are shown in the embodiment illustrated in FIG. 3, in other embodiments the funnel 332 can include more or fewer pores, such as at least 1, 5, 10, 20, 30, 50, 100, a number therebetween, or another suitable number of pores 333. Individual ones of the pores 333 can have a cross-sectional dimension (e.g., width) or area of between about 100 μm and about 500 such as less than or equal to 150 or another suitable dimension or area. Referring to FIGS. 2 and 3 together, the pores 333 of the funnel 332 can be sized and shaped to (i) substantially allow blood and/or other fluids within the left atrial appendage LAA to flow through the funnel 332 and (ii) inhibit clot material 201 within the left atrial appendage LAA from flowing through the funnel 332.

The funnel 332 can be configured to inhibit the clot material 201 (FIG. 2) within the left atrial appendage LAA from embolizing and flowing through the heart H and vasculature of a patient during a clot treatment and/or removal procedure. For example, during the procedure described in detail with reference to FIGS. 1A, 1B, and 2, the device 330 can be deployed from the catheter 120 and used to inhibit or prevent the clot material 201 from passing out of the ostium O of the left atrial appendage LAA (e.g., embolizing) during aspiration of the catheter 120. More specifically, the distal edge 336 of the funnel 332 can be aligned with and/or positioned against the ostium O such that the funnel 332 can at least partially cover/occlude the ostium O and “catch” the clot material 201 when the clot material 201 breaks free from the left atrial appendage LAA, such as in response to aspiration. Additionally or alternatively, the funnel 332 can be positioned/deployed at least partially within the left atrial appendage LAA, such that at least a portion of the funnel 332 (e.g., the distal edge 336) can contact an interior surface of the left atrial appendage LAA.

In some embodiments, in addition or alternatively to aspirating the lumen 124 of the catheter 120, a lumen of the elongate member 338 can be used to aspirate the clot material 201. For example, a pressure source and tubing system similar or identical to the pressure source 104 and tubing assembly 110 of FIG. 1A can be coupled to the elongate member 338 and used to aspirate the elongate member 338. In these and other embodiments, one or more other clot treatment and/or removal devices can be positioned within and/or delivered through the elongate member 338 and/or the catheter 120 to aspirate and/or engage the clot material 201.

Referring to FIG. 4, the device 430 can include a funnel 432 generally similar to the funnel 332 of FIG. 3. For example, the funnel 432 can be configured to “catch” the clot material 201 and/or at least partially or fully prevent the clot material from embolizing, as described previously with reference to FIG. 3. In the illustrated embodiment, however, the funnel 432 has a shorter length and is positioned such that a distal edge 436 of the funnel 432 is generally coplanar with the distal tip 340 of the elongate member 338, and/or such that the distal tip 340 extends distally beyond the distal edge 436 of the funnel 432. More specifically, for example, a proximal end portion 434a of the funnel 432 can be coupled to the elongate member 338 proximal of the distal tip 340. Accordingly, referring to FIGS. 2 and 4 together, when used during a clot treatment procedure to treat the clot material 201 within the ostium O of the left atrial appendage LAA, the distal tip 340 of the elongate member 338 can extend through the ostium O of the left atrial appendage LAA and/or otherwise be positioned closer to the clot material 201 located therein. In some aspects, positioning the distal tip 340 of the elongate member 338 closer to the clot material 201 is expected to improve the suction force applied to the clot material 201 and/or increase the amount of clot material 201 removed during a given aspiration pulse (e.g., applied through the elongate member 338). In other embodiments, however, the distal edge 436 can be positioned proximally or distally relative to the distal tip 340.

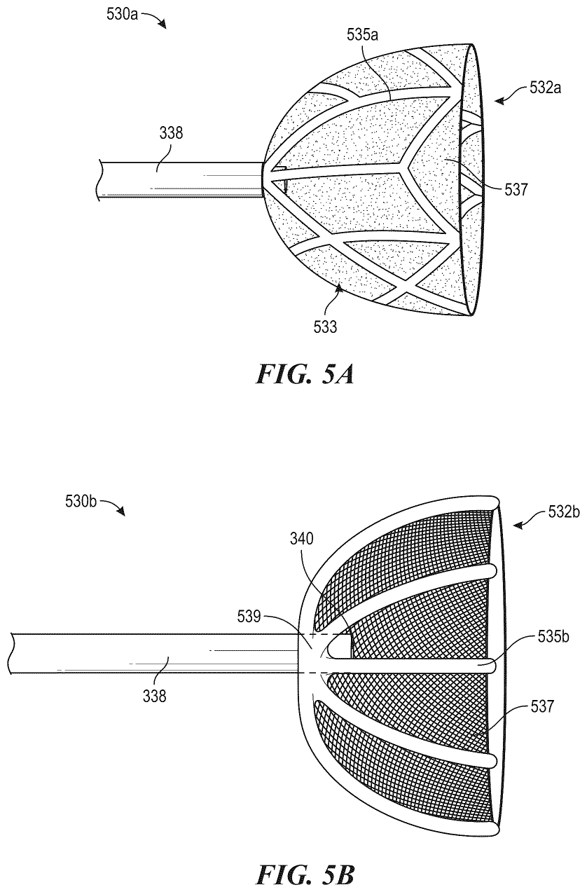

Referring to FIG. 5A, the device 530a can include a funnel 532a having a plurality of support elements or struts 535a and a filtering layer 537 coupled thereto. The funnel 532a can be coupled to the elongate member 338. Individual ones of the struts 535a can be formed from a shape memory material, such as nitinol, and can be interconnected with one or more of the other struts 535a. In at least some embodiments, the funnel 532a can be self-expanding and/or configured to automatically transition from a low-profile delivery state to an expanded state (shown in FIG. 5A). The filtering layer 537 can cover one or more or all of the struts 535a and can be formed from expanded polytetrafluoroethylene (ePTFE), polytetrafluoroethylene (PTFE), polyethylene terephthalate (PET) mesh, a silicone coating, and/or another suitable material. Additionally, the filtering layer 537 can include/define multiple pores 533, such that the funnel 532a can be configured to (i) substantially allow blood and/or other fluid(s) within the left atrial appendage LAA to flow through the funnel 532a and (ii) inhibit the clot material 201 within the left atrial appendage LAA from flowing through the funnel 532a.

Referring to FIG. 5B, the device 530b can include a funnel 532b generally similar to the funnel 532a of FIG. 5A. In the illustrated embodiment, the funnel 532b includes one or more struts 535b that extend radially outwardly in a distal direction from a central hub 539. The central hub can be coupled to the elongate member 338, such that each of the struts 535b can extend distally beyond the distal tip 340 of the elongate member 338 in a direction generally parallel to a longitudinal axis of the elongate member 338. Individual ones of the struts 535b can be formed from a shape memory material, such as nitinol, such that the funnel 532b can be self-expanding and/or configured to automatically transition from a low-profile delivery state to an expanded state (shown in FIG. 5B). The filtering layer 537 can cover one or more or all of the struts 535b.

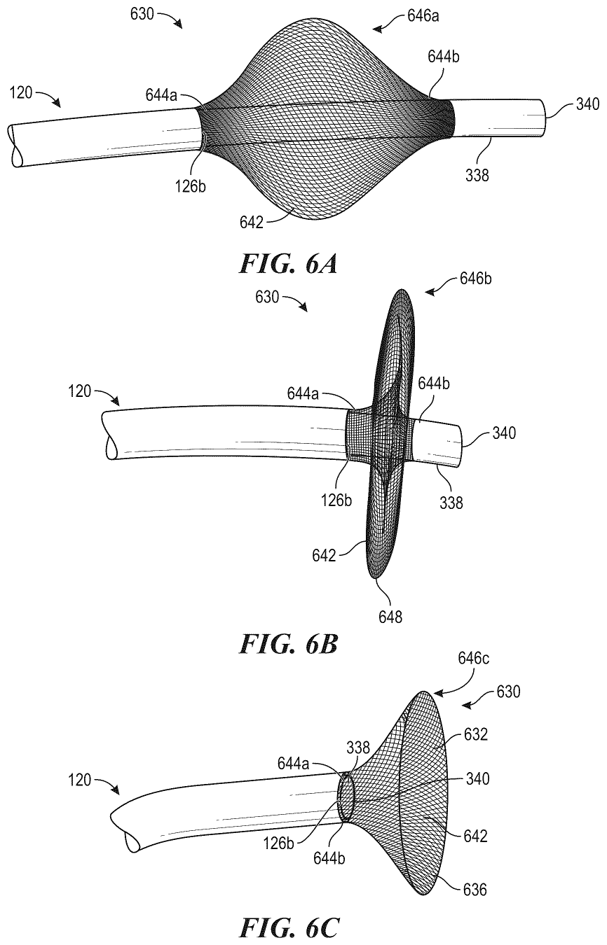

Referring to FIGS. 6A-6C together, the device 630 includes an adjustable structure 642 having a first (e.g., proximal) end portion 644a and a second (e.g., distal) end portion 644b. The second end portion 644b can be coupled to the elongate member 338, for example, at or near the distal tip 340. The first end portion 644a can be coupled to a catheter, such as the catheter 120 shown in FIGS. 6A-6C, or another suitable catheter. In the illustrated embodiment, the adjustable structure 642 includes a shape-memory wire structure. In other embodiments, the adjustable structure 642 can comprise a mesh or other suitable structure. The elongate member 338 can be slidably disposed within the catheter 120, such that one or both of the elongate member 338 and the catheter 120 can be moved relative to one another.

Relative movement between the elongate member 338 and the catheter 120 can transition the adjustable structure 642 between multiple shapes or states, such as a first state 646a (e.g., a spherical, disc, or orb shape) shown in FIG. 6A, a second state 646b (e.g., a flat or disk shape shown in FIG. 6B), and a third state 646c (e.g., a conical or funnel shape) shown in FIG. 6C. For example, moving the elongate member 338 relative to the catheter 120, and/or moving the catheter 120 relative to the elongate member 338, can move the first and second end portions 644a-b of the adjustable structure 642 relative to one another and cause the adjustable structure 642 to change shape. In the first state 646a, the adjustable structure 642 can be curved or arcuate and the first and second end portions 644a-b can be generally spaced apart from each other. In the second state 646b, the adjustable structure 642 can include an apex region 648 that defines a maximum outer dimension (e.g., width, diameter, and the like) of the adjustable structure 642, e.g., a maximum outer dimension for all or a subset of the various states 646a-c. For example, the diameter of the adjustable structure 642 in the second state 646b can be greater than the diameter of the adjustable structure 642 in the first state 646a and/or the third state 646c. In the third state 646c, the adjustable structure 642 can define a funnel 632 including a distal edge 636 defined by a fold in the adjustable structure 642. The fold in the adjustable structure 642 can be formed by positioning the second end portion 644b within or near to the first end portion 644a, as described in further detail below.

The adjustable structure 642 can be transitioned from the first state 646a to the second state 646b by decreasing the distance between the first and second end portions 644a-b, for example, by moving the distal tip 340 of the elongate member 338 toward the catheter 120 and/or moving the catheter 120 toward the distal tip 340. The adjustable structure 642 can be transitioned from the second state 646b to the third state 646c by continuing to decrease the distance between the first and second end portions 644a-b, for example, until the first and second end portions 644a-b at least partially overlap each other and/or until the distal tip 340 is positioned within the catheter 120. The adjustable structure 642 can be transitioned from the third state 646c to the second state 646b and/or the first state 646a by increasing the distance between the first and second end portions 644a-b. In these and other embodiments, the adjustable structure 642 can be configured to transition to one or more additional states/shapes other than the shapes 646a-c illustrated in FIGS. 6A-6C. For example, the adjustable structure 642 can be transitioned to a low-profile delivery state by further increasing the distance between the first and second end portions 644a-b to thereby advance the distal tip 340 further distally away from the catheter 120 (e.g., further distally advancing the distal tip 340 away/outwardly from the distal terminus 126b of the catheter 120) to cause the adjustable structure 642 to lay substantially flat against the outer surface of the elongate member 338.

Referring to FIGS. 2 and 6A, 6B, and 6C together, the shape of the adjustable structure 642 as shown, for example, by the first state 646a, the second state 646b, and the third state 646c, can be adjusted in vivo based on the specific geometry of a given patient's left atrial appendage LAA. In some aspects of the present technology, this can further improve the treatment of the clot material 201. For example, the first state 646a can allow for deeper insertion of the distal tip 340 into the left atrial appendage LAA and/or can center the elongate member 338 within the left atrial appendage LAA, both of which can improve aspiration of the clot material 201 from within the left atrial appendage LAA via the catheter 120 and/or the elongate member 338. In the second state 646b, the greater outer dimension (e.g., diameter, width, radius) of the adjustable structure 642 can allow the adjustable structure 642 to cover/occlude many differently-sized left atrial appendages LAA. In the third state 646c, the distance between the distal tip 340 of the elongate member and the distal edge 636 of the funnel 632 can reduce or prevent application of aspiration forces (e.g., suction) on the wall of the left atrial appendage LAA and/or other portions of the patient's anatomy.

FIG. 7 is a side view of a clot treatment and/or removal device 750 (“device 750”) positioned within the left atrial appendage LAA in accordance with embodiments of the present technology. In some embodiments, the device 750 can be used in the clot treatment system 100 described in detail with reference to FIGS. 1A-6. For example, the device 750 can be advanced through and/or retracted into the elongate member 338 and/or the catheter 120. Generally, the device 750 can include at least some aspects that are at least generally similar or identical in structure and/or function to one or more of the devices described in detail in U.S. Pat. No. 10,098,651, titled “DEVICES AND METHODS FOR TREATING VASCULAR OCCLUSION,” and filed Apr. 26, 2017, which is hereby incorporated by reference in its entirety.

In the illustrated embodiment, the device 750 is a mechanical thrombectomy device including a braided or laser-cut structure coupled to a tether 752. The device 750 can be formed at least partially from a shape memory material, such as nitinol. Referring to FIGS. 2 and 7 together, the device 750 can be inserted through the ostium O into the left atrial appendage LAA to capture/engage the clot material 201 engaged therein. Then, the tether 752 can be used to withdraw the device 750 from the left atrial appendage LAA such that the device 750 can remove/break free at least a portion of the clot material 201. In the illustrated embodiment, for example, at least a portion of the device 430 of FIG. 4, such as at least a portion of the funnel 432 and/or the distal edge 436 thereof, is positioned against the ostium O and the device 750 has been inserted into the left atrial appendage LAA through the elongate member 338. After positioning the device 750 in the left atrial appendage LAA, the device 750 can be withdrawn into the elongate member 338. The device 430 (e.g., the funnel 432) can capture any of the clot material 201 that breaks free before, during, and/or after mechanical engagement of the device 750 with the clot material 201, and/or otherwise prevent at least a portion of the clot material 201 form embolizing.

In some embodiments, a clot capture device in accordance with the present technology can be used with the clot removal system of FIG. 1A and positioned elsewhere in the vasculature of a patient downstream of the left atrial appendage to capture clot material removed or broken free from the left atrial appendage LAA of a patient, for example, to thereby inhibit or prevent the clot material from causing an embolic event (e.g., embolism, stroke, and the like). For example, FIG. 8 is a side cross-sectional view of a heart H of a patient including a clot capture device 830 having a funnel 832 configured to be positioned downstream from the heart H, such as within the aorta (e.g., the ascending aorta and/or the descending aorta). The funnel 832 can include at least some aspects that are generally similar or identical in structure and/or function to one or more of the funnels 332, 432, 532a, 532b, 632 described previously herein. The device 830 can be used in conjunction with one or more of the other devices 330-630, 750 described herein, for example, while one or more of the other devices 330-630, 750 are positioned within the heart H. Additionally, or alternatively, the device 830 can be used with an aspiration catheter system, such as the system 100 and/or another suitable aspiration catheter system, to aspirate at least a portion of the clot material 201 that exits the left atrial appendage LAA. Generally, the device 830 can include at least some aspects that are generally similar or identical to one or more of the clot treatment systems described in detail in U.S. patent application Ser. No. 17/339,663, titled “RECAPTURABLE FUNNEL CATHETERS, AND ASSOCIATED SYSTEMS AND METHODS,” and filed Jun. 4, 2021, which is incorporated herein by reference in its entirety.

In the illustrated embodiment, the funnel 832 is coupled to an elongate member 838 that has been inserted through the descending aorta toward the left ventricle LV. Accordingly, the funnel 832 can catch any of the clot material 201 that exits the left atrial appendage LAA (FIG. 2) and flows downstream through the left atrium LA and/or the left ventricle LV. The funnel 832 can be configured to transition between a low-profile delivery state and an expanded state (shown in FIG. 8). In the low-profile delivery state, the funnel 832 can be configured for insertion through one or more of the patient's arteries, such as the femoral artery or the radial artery. For example, in the low profile delivery state, the funnel 832 can have an outer dimension (e.g., diameter) between about 6 Fr to 8 Fr. In the expanded state, the funnel 832 can be configured to span all or a portion of the inner width of the vessel (e.g., the aorta) within which the funnel 832 is positioned. For example, in the expanded state, the funnel 832 can have outer dimension (e.g., diameter) between about 30 mm to about 40 mm, or another suitable outer dimension.

In some embodiments, at least a portion of the clot treatment system 100 can be used to remove clot material from one or more portions or regions of the patient's anatomy other than the left atrial appendage LAA. For example, FIG. 9 is a side cross-sectional view of a heart H of a patient with the shaped portion 128 of the catheter 120 of FIGS. 1A and 1B positioned in accordance with embodiments of the present technology. At least part of the shaped portion 128 can be positioned within the left atrium LA via the opening 203 formed in the septum between the left atrium LA and the right atrium RA. In the illustrated embodiment, for example, the shaped portion 128 extends into the left atrium LA from the right atrium RA. In this position, the catheter 120 can be used to aspirate all, or at least a portion, of the clot material 201 within the left atrium LA. The catheter 120 can be advanced, rotated, and/or otherwise moved to position the catheter 120 at a desired position within the left atrium LA proximate to the clot material 201.

FIG. 10 is a side cross-sectional view of a heart H of a patient with the clot capture device 330 of FIG. 3 positioned in accordance with embodiments of the present technology. The clot capture device 330 (e.g., the funnel 332) can be positioned between a first anatomical location or body region (e.g., the left atrium LA, the right atrium RA, the septum S) and a second anatomical location or body region (e.g., a different one or portion of the left atrium LA, the right atrium RA, and the septum S), e.g., to help guide the clot material 210 into the catheter 120 and/or inhibit or even prevent the clot material 210 in the first anatomical location from flowing toward and/or into the second anatomical location. For example, in the expanded state, the funnel 332 can be positioned through and/or in contact with the septum S between the left atrium LA and the right atrium RA. In the illustrated embodiment, the funnel 332 extends through the septum S into the left atrium LA to help guide the clot material 210 into the catheter, e.g., during aspiration. Additionally, or alternatively, the funnel 332 presses against the septum S so that all, or at least a portion, of fluid flow between the left atrium LA and the right atrium RA through the septum S passes through the funnel 332. This, in turn, can inhibit or even prevent the clot material 201 in the left atrium LA from flowing toward and/or into the right atrium, e.g., unless or until the clot material 201 is aspirated by the catheter 120. In some embodiments, the funnel 332 can be transitioned from the low-profile delivery state toward and/or to the expanded state with at least a portion of the funnel 332 positioned through the opening 203. Additionally, or alternatively, the funnel 332 can be positioned through the opening 203, transitioned toward and/or to the expanded state, and then moved toward the right atrium RA, e.g., so that at least part of the proximal end portion 334a is positioned within the opening 203 or the right atrium RA. With the clot capture device 330 positioned at least partially within the left atrium LA, the catheter 120 can be used to aspirate all, or at least a portion, of the clot material 201 from within the left atrium LA. In other embodiments, the funnel 332 can be positioned fully inside the left atrium LA, e.g., not within or in contact with the septum S. Although FIG. 10 illustrated the clot capture device 330, the other clot capture devices 430-630 described herein can also be positioned and/or used in an at least generally similar or identical manner to the clot capture device 330.

### Examples

Several aspects of the present technology are set forth in the following examples:
1. A clot treatment system for the treatment of clot material within a left atrial appendage of a human patient, comprising:
a catheter including— a proximal portion; and
   a shaped distal portion curved relative to the proximal portion;
a pressure source fluidly coupled to the catheter and configured to aspirate at least a portion of the clot material from the left atrial appendage via the shaped distal portion; and
a clot capture device configured to be positioned at least partially over an opening of the left atrial appendage to inhibit any of the clot material from leaving the left atrial appendage outside the catheter.
2. The clot treatment system of example 1 wherein the clot capture device includes an elongate member and a funnel coupled to the elongate member, wherein the funnel is configured to transition between a low-profile delivery state and an expanded state, and wherein the clot capture device is positionable within the catheter and configured to move relative to the catheter to transition the funnel between the low-profile delivery state and the expanded state.
3. The clot treatment system of example 2 wherein the elongate member includes a distal tip, wherein the funnel includes a distal edge, and wherein the distal tip of the elongate member extends distally beyond the distal edge of the funnel.
4. The clot treatment system of example 2 wherein the elongate member includes a distal tip, wherein the funnel includes a distal edge, and wherein the distal tip is coplanar with the distal edge.
5. The clot treatment system of any of examples 2-4 wherein the funnel includes a filtering layer and one or more shape-memory struts.
6. The clot treatment system of any one of examples 1-5 wherein the clot capture device includes an adjustable structure configured to transition between a plurality of shapes.
7. The clot treatment system of example 6 wherein the adjustable structure is configured to transition between an orb shape, a disk shape, and a funnel shape.
8. The clot treatment system of example 6 or example 7, wherein the clot capture device includes an elongate member configured to be positioned within the catheter, wherein the adjustable structure includes a first end portion coupled to the catheter and a second end portion coupled to the elongate member, and wherein the elongate member and the catheter are configured to move relative to one another to change the shape of the adjustable structure.
9. The clot treatment system of example 8 wherein the adjustable structure is configured to transition from an orb shape to a funnel shape in response to proximal movement of the elongate member relative to the catheter.
10. The clot treatment system of any one of examples 1-9 wherein the shaped distal portion includes:
an intermediate portion adjacent to and distal from the proximal portion; and
a distal portion adjacent to and distal from the intermediate portion;
wherein—
   the intermediate portion is curved relative to the proximal portion in a first direction, and
   the distal portion is curved relative to the proximal portion in a second direction.
11. The clot treatment system of example 10 wherein the first direction is coplanar with a longitudinal axis of the proximal portion, and wherein the second direction is perpendicular to the first direction.
12. The clot treatment system of example 10 or example 11 wherein the proximal portion and the intermediate portion define a plane, and wherein the second direction is perpendicular to the plane.
13. The clot treatment system of any or examples 10-12 wherein the intermediate portion is curved at a first angle about a first radius, and wherein the distal portion is curved at a second angle about a second radius.
14. The clot treatment system of example 13 wherein—
the first angle is 90 degrees;
the first radius is 2 inches;
the second angle is 30 degrees; and the second radius is 1.25 inches.
15. The clot treatment system of any one of examples 1-14, wherein the shaped distal portion further includes a distalmost tip portion configured to be aligned with or positioned within the left atrial appendage.
16. The clot treatment system of any one of examples 1-15 wherein the clot capture device is a first clot capture device, the clot treatment system further comprising a second clot capture device configured to be positioned downstream from the left atrial appendage.
17. The clot treatment system of example 16 wherein the second clot capture device is configured to be positioned in an aorta of the patient.
18. The clot treatment system of any one of examples 1-17 wherein the clot capture device is configured to be positioned at least partially within the left atrial appendage.
19. A method for treatment of clot material within a left atrial appendage of a human patient, the method comprising:
positioning a shaped distal portion of a catheter proximate to the clot material;
positioning a clot capture device to at least partially cover an opening of left atrial appendage;
coupling a pressure source to the catheter via a fluid control device, wherein (a) opening of the fluid control device fluidly connects the pressure source to the catheter and (b) closing of the fluid control device fluidly disconnects the pressure source from the catheter;
activating the pressure source to generate a vacuum; and
applying the vacuum to the catheter to thereby aspirate at least a portion of the clot material into the shaped distal portion of the catheter.
20. The method of example 19 wherein positioning the shaped distal portion includes transeptally inserting the shaped distal portion into the left atrium of a heart of the human patient from the right atrium of the heart.
21. The method of example 20 wherein positioning the clot capture device includes aligning the clot capture device with the left atrial appendage.
22. The method of any one of examples 19-21 wherein positioning the clot capture device includes positioning the clot capture device to contact at least a portion of an ostium of the left atrial appendage.
23. The method of any of examples 19-22 wherein positioning the clot capture device includes positioning a funnel of the clot capture device relative to the left atrial appendage such that the funnel (i) at least partially prevents the clot material from leaving the left atrial appendage and (ii) substantially allows blood within the left atrial appendage to flow out of the left atrial appendage through the funnel.
24. The method of any one of examples 19-23 wherein positioning the clot capture device includes positioning an elongate member of the clot capture device at least partially within the left atrial appendage.
25. The method of example 24 wherein the elongate member is positioned within the catheter and fluidly coupled to the pressure source such that opening the fluid control device to apply the vacuum to the catheter includes opening the fluid control device to apply the vacuum to the elongate member to thereby aspirate at least the portion of the clot material.
26. The method of any one of examples 19-25 wherein positioning the clot capture device includes extending the clot capture device from within the catheter.
27. The method of any one of examples 19-26 wherein positioning the clot capture device includes transitioning the clot capture device from a low-profile delivery state to an expanded state.
28. The method of any one of examples 19-26 wherein positioning the clot capture device includes changing a shape of the clot capture device by moving an elongate member of the clot capture device relative to the catheter.
29. The method of example 28 wherein moving the elongate member relative to the catheter includes moving the elongate member proximally or distally relative to the catheter.
30. The method of example 28 or example 29 wherein changing the shape of the clot capture device includes transitioning the clot capture device between at least two of an orb shape, a disk shape, and a funnel shape.
31. The method of any one of examples 19-30 wherein the clot capture device is a first clot capture device, the method further comprising positioning a second clot capture device downstream from the left atrial appendage.
32. The method of any one of examples 19-31, further comprising positioning a clot treatment device to at least partially contact the clot material.
33. The method of example 32 wherein positioning the clot treatment device includes inserting the clot treatment device through the shaped distal portion of the catheter or the clot capture device.
34. The method of example 32 or example 33 wherein positioning the clot treatment device includes inserting the clot treatment device at least partially into a left atrial appendage of the patient.
35. The method of any one of examples 32-34 wherein positioning the clot treatment device includes positioning a mechanical thrombectomy device to contact at least a portion of the clot material.
36. The method of any one of examples 19-35, wherein activating the pressure source to generate the vacuum includes activating the pressure source to generate the vacuum while the fluid control device is closed, and wherein applying the vacuum to the catheter includes opening the fluid control device.
37. The method of any one of examples 19-35 wherein activating the pressure source to generate the vacuum includes activating the pressure source to generate the vacuum while the fluid control device is open to thereby apply the vacuum to the catheter and aspirate at least the portion of the clot material.
38. A clot treatment system for the treatment of clot material within a heart of a human patient, comprising:
a catheter including—
   a proximal portion; and
   a shaped distal portion curved relative to the proximal portion;
a pressure source fluidly coupled to the catheter and configured to aspirate at least a portion of the clot material from the heart via the shaped distal portion; and
a clot capture device configured to be positioned at least partially within the heart to inhibit any of the clot material from leaving the heart outside the catheter.
39. The clot treatment system of example 38 wherein the clot capture device is configured to at least partially inhibit the clot material from causing an embolic event.
40. The clot treatment system of example 38 or example 39 wherein the heart includes a left atrium, and wherein the clot capture device is configured to be positioned at least partially within the left atrium of the heart.
41. A clot treatment system for the treatment of clot material within a heart of a human patient, the clot treatment system comprising:
a first catheter carrying a first clot capture device configured to be positioned at a first location downstream from the clot material and to at least partially prevent downstream movement of at least a first portion of the clot material;
a second catheter carrying a second clot capture device configured to be positioned at a second location downstream from the first clot capture device and to at least partially prevent further downstream movement of at least a second portion of the clot material; and
a pressure source fluidly coupled to the first catheter or the second catheter and configured to aspirate at least a third portion of the clot material.
42. The clot treatment system of example 41 wherein the first location is at or near a left atrial appendage of the human patient.
43. The clot treatment system of example 41 or example 42 wherein the second location is at least partially within a left atrium, a left ventricle, or an aorta of the human patient.
44. The clot treatment system of example 41 wherein the first location is at least partially within a left atrium of the human patient.
45. The clot treatment system of example 44 wherein the second location is at least partially within a left ventricle or an aorta of the human patient.
46. The clot treatment system of any one of examples 41-45 wherein the first clot capture device or the second clot capture device includes a funnel.
47. The clot treatment system of any one of examples 41-46 wherein the first clot capture device or the second clot capture device is configured to be transition between at least two different shapes.
48. The clot treatment system of any one of examples 41-47 wherein:
the first clot capture device includes a first distal edge, the first catheter includes a first distal tip positioned proximally from the first distal edge, and/or
the second clot capture device includes a second distal edge and the second catheter includes a second distal tip positioned proximally from the second distal edge.
49. The clot treatment system of any one of examples 41-47 wherein:
the first clot capture device includes a first distal edge and the first catheter includes a first distal tip positioned distally from the first distal edge, and/or
the second clot capture device includes a second distal edge and the second catheter includes a second distal tip positioned distally from the second distal edge.
50. The clot treatment system of any one of examples 41-47 wherein:
the first clot capture device includes a first distal edge and the first catheter includes a first distal tip positioned coplanar with the first distal edge, and/or
the second clot capture device includes a second distal edge and the second catheter includes a second distal tip positioned coplanar with the second distal edge.
51. The clot treatment system of any one of examples 41-50 wherein the third portion of the clot material includes at least part or all of the first portion or the second portion of the clot material.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A clot treatment system for the treatment of clot material within a left atrial appendage of a human patient, comprising:
a catheter including—
a proximal portion; and
a shaped distal portion curved relative to the proximal portion;
a pressure source fluidly coupled to the catheter and configured to aspirate at least a portion of the clot material from the left atrial appendage via the shaped distal portion; and
a clot capture device configured to be positioned at least partially over an opening of the left atrial appendage to inhibit any of the clot material from leaving the left atrial appendage outside the catheter.

2. The clot treatment system of claim 1 wherein the clot capture device includes an elongate member and a funnel coupled to the elongate member, wherein the funnel is configured to transition between a low-profile delivery state and an expanded state, and wherein the clot capture device is positionable within the catheter and configured to move relative to the catheter to transition the funnel between the low-profile delivery state and the expanded state.

3. The clot treatment system of claim 2 wherein the elongate member includes a distal tip, wherein the funnel includes a distal edge, and wherein the distal tip of the elongate member extends distally beyond the distal edge of the funnel.

4. The clot treatment system of claim 2 wherein the elongate member includes a distal tip, wherein the funnel includes a distal edge, and wherein the distal tip is coplanar with the distal edge.

5. The clot treatment system of claim 2 wherein the funnel includes a filtering layer and one or more shape-memory struts.

6. The clot treatment system of claim 1 wherein the clot capture device includes an adjustable structure configured to transition between a plurality of shapes.

7. The clot treatment system of claim 6 wherein the adjustable structure is configured to transition between an orb shape, a disk shape, and a funnel shape.

8. The clot treatment system of claim 6, wherein the clot capture device includes an elongate member configured to be positioned within the catheter, wherein the adjustable structure includes a first end portion coupled to the catheter and a second end portion coupled to the elongate member, and wherein the elongate member and the catheter are configured to move relative to one another to change the shape of the adjustable structure.

9. The clot treatment system of claim 8 wherein the adjustable structure is configured to transition from an orb shape to a funnel shape in response to proximal movement of the elongate member relative to the catheter.

10. The clot treatment system of claim 1 wherein the shaped distal portion includes:
an intermediate portion adjacent to and distal from the proximal portion; and
a distal portion adjacent to and distal from the intermediate portion;
wherein—
the intermediate portion is curved relative to the proximal portion in a first direction, and
the distal portion is curved relative to the proximal portion in a second direction.

11. The clot treatment system of claim 10 wherein the first direction is coplanar with a longitudinal axis of the proximal portion, and wherein the second direction is perpendicular to the first direction.

12. The clot treatment system of claim 10 wherein the proximal portion and the intermediate portion define a plane, and wherein the second direction is perpendicular to the plane.

13. The clot treatment system of claim 10 wherein the intermediate portion is curved at a first angle about a first radius, and wherein the distal portion is curved at a second angle about a second radius.

14. The clot treatment system of claim 13 wherein—
the first angle is 90 degrees;
the first radius is 2 inches;
the second angle is 30 degrees; and
the second radius is 1.25 inches.

15. The clot treatment system of claim 1, wherein the shaped distal portion further includes a distalmost tip portion configured to be aligned with or positioned within the left atrial appendage.

16. The clot treatment system of claim 1 wherein the clot capture device is a first clot capture device, the clot treatment system further comprising a second clot capture device configured to be positioned downstream from the left atrial appendage.

17. The clot treatment system of claim 16 wherein the second clot capture device is configured to be positioned in an aorta of the patient.

18. The clot treatment system of claim 1 wherein the clot capture device is configured to be positioned at least partially within the left atrial appendage.

19. A method for treatment of clot material within a left atrial appendage of a human patient, the method comprising:
positioning a shaped distal portion of a catheter proximate to the clot material;
positioning a clot capture device to at least partially cover an opening of the left atrial appendage;
coupling a pressure source to the catheter via a fluid control device, wherein (a) opening the fluid control device fluidly connects the pressure source to the catheter and (b) closing the fluid control device fluidly disconnects the pressure source from the catheter;
activating the pressure source to generate a vacuum; and
applying the vacuum to the catheter to thereby aspirate at least a portion of the clot material into the shaped distal portion of the catheter.

20. The method of claim 19 wherein positioning the shaped distal portion includes transeptally inserting the shaped distal portion into the left atrium of a heart of the human patient from the right atrium of the heart.

21. The method of claim 20 wherein positioning the clot capture device includes aligning the clot capture device with the left atrial appendage.

22. The method of claim 19 wherein positioning the clot capture device includes positioning the clot capture device to contact at least a portion of an ostium of the left atrial appendage.

23. The method of claim 19 wherein positioning the clot capture device includes positioning a funnel of the clot capture device relative to the left atrial appendage such that the funnel (i) at least partially prevents the clot material from leaving the left atrial appendage and (ii) substantially allows blood within the left atrial appendage to flow out of the left atrial appendage through the funnel.

24. The method of claim 19 wherein positioning the clot capture device includes positioning an elongate member of the clot capture device at least partially within the left atrial appendage.

25. The method of claim 24 wherein the elongate member is positioned within the catheter and fluidly coupled to the pressure source such that opening the fluid control device to apply the vacuum to the catheter includes opening the fluid control device to apply the vacuum to the elongate member to thereby aspirate at least the portion of the clot material.

26. The method of claim 19 wherein positioning the clot capture device includes extending the clot capture device from within the catheter.

27. The method of claim 19 wherein positioning the clot capture device includes transitioning the clot capture device from a low-profile delivery state to an expanded state.

28. The method of claim 19 wherein positioning the clot capture device includes changing a shape of the clot capture device by moving an elongate member of the clot capture device relative to the catheter.

29. The method of claim 28 wherein moving the elongate member relative to the catheter includes moving the elongate member proximally or distally relative to the catheter.

30. The method of claim 28 wherein changing the shape of the clot capture device includes transitioning the clot capture device between at least two of an orb shape, a disk shape, and a funnel shape.

31. The method of claim 19 wherein the clot capture device is a first clot capture device, the method further comprising positioning a second clot capture device downstream from the left atrial appendage.

32. The method of claim 19, further comprising positioning a clot treatment device to at least partially contact the clot material.

33. The method of claim 32 wherein positioning the clot treatment device includes inserting the clot treatment device through the shaped distal portion of the catheter or the clot capture device.

34. The method of claim 32 wherein positioning the clot treatment device includes inserting the clot treatment device at least partially into a left atrial appendage of the patient.

35. The method of claim 32 wherein positioning the clot treatment device includes positioning a mechanical thrombectomy device to contact at least a portion of the clot material.

36. The method of claim 19, wherein activating the pressure source to generate the vacuum includes activating the pressure source to generate the vacuum while the fluid control device is closed, and wherein applying the vacuum to the catheter includes opening the fluid control device.

37. The method of claim 19 wherein activating the pressure source to generate the vacuum includes activating the pressure source to generate the vacuum while the fluid control device is open to thereby apply the vacuum to the catheter and aspirate at least the portion of the clot material.

38. A clot treatment system for the treatment of clot material within a heart of a human patient, comprising:
a catheter including—
a proximal portion; and
a shaped distal portion curved relative to the proximal portion;
a pressure source fluidly coupled to the catheter and configured to aspirate at least a portion of the clot material from the heart via the shaped distal portion; and
a clot capture device configured to be positioned at least partially within the heart to inhibit any of the clot material from leaving the heart outside the catheter.

39. The clot treatment system of claim 38 wherein the clot capture device is configured to at least partially inhibit the clot material from causing an embolic event.

40. The clot treatment system of claim 38 wherein the heart includes a left atrium, and wherein the clot capture device is configured to be positioned at least partially within the left atrium of the heart.

41. A clot treatment system for the treatment of clot material within a heart of a human patient, the clot treatment system comprising:
a first catheter carrying a first clot capture device configured to be positioned at a first location downstream from the clot material and to at least partially prevent downstream movement of at least a first portion of the clot material;
a second catheter carrying a second clot capture device configured to be positioned at a second location downstream from the first clot capture device and to at least partially prevent further downstream movement of at least a second portion of the clot material; and
a pressure source fluidly coupled to the first catheter or the second catheter and configured to aspirate at least a third portion of the clot material.

42. The clot treatment system of claim 41 wherein the first location is at or near a left atrial appendage of the human patient.

43. The clot treatment system of claim 41 wherein the second location is at least partially within a left atrium, a left ventricle, or an aorta of the human patient.

44. The clot treatment system of claim 41 wherein the first location is at least partially within a left atrium of the human patient.

45. The clot treatment system of claim 44 wherein the second location is at least partially within a left ventricle or an aorta of the human patient.

46. The clot treatment system of claim 41 wherein the first clot capture device or the second clot capture device includes a funnel.

47. The clot treatment system of claim 41 wherein the first clot capture device or the second clot capture device is configured to be transition between at least two different shapes.

48. The clot treatment system of claim 41 wherein:
the first clot capture device includes a first distal edge, the first catheter includes a first distal tip positioned proximally from the first distal edge, and/or
the second clot capture device includes a second distal edge and the second catheter includes a second distal tip positioned proximally from the second distal edge.

49. The clot treatment system of claim 41 wherein:
the first clot capture device includes a first distal edge and the first catheter includes a first distal tip positioned distally from the first distal edge, and/or
the second clot capture device includes a second distal edge and the second catheter includes a second distal tip positioned distally from the second distal edge.

50. The clot treatment system of claim 41 wherein:
the first clot capture device includes a first distal edge and the first catheter includes a first distal tip positioned coplanar with the first distal edge, and/or
the second clot capture device includes a second distal edge and the second catheter includes a second distal tip positioned coplanar with the second distal edge.

51. The clot treatment system of claim 41 wherein the third portion of the clot material includes at least part or all of the first portion or the second portion of the clot material.
